(19) United States
(12) Patent Application Publication
(10) Pub. No.: US 2025/0308630 A1
(43) Pub. Date: Oct. 2, 2025

(54) Method for establishing a tumor neoantigen database and its application

(71) Applicant: National Taiwan University, Taipei City (TW)

(72) Inventors: Eric-Y Chuang, Taipei City (TW); Chien-Yueh Lee, Taipei City (TW); Tzu-Pin Lu, Taipei City (TW); Chia-Yu Sung, Taipei City (TW); Chia-Hsin Wu, Taipei City (TW); Yu-Hsuan Tseng, Taipei City (TW); Mong-Hsun Tsai, Taipei City (TW); Liang-Chuan Lai, Taipei City (TW); Kun-San Chao, Taipei City (TW); Chih-Yang Huang, Taipei City (TW)

(21) Appl. No.: 18/623,066

(22) Filed: Apr. 1, 2024

(51) Int. Cl. G16B 20/50; G16B 15/30; G16B 35/10; G16B 35/20; G16B 50/30

(52) U.S. Cl. CPC G16B 20/50; G16B 15/30; G16B 35/10; G16B 35/20; G16B 50/30

(57) ABSTRACT

A method for establishing a tumor neoantigen database is and processes for identifying the mutated tumor-specific antigens (mTSAs) and aberrantly expressed tumor-specific antigens (aeTSAs) are disclosed. In particular, the tumor neoantigen database is used for predicting tumor variants of clinical samples.

### TECHNICAL FIELD

The invention relates to a method for establishing a tumor neoantigen database and processes for identifying the mutated tumor-specific antigens (mTSAs) and aberrantly expressed tumor-specific antigens (aeTSAs), respectively. In particular, the tumor neoantigen database is used for predicting tumor variants of clinical samples.

### BACKGROUND

In 2020, there were 19.3 million new cancer cases worldwide, with 10 million cancer-related deaths. The Asian region accounted for over 50% of global cancer deaths. In Taiwan, there were over 110,000 newly diagnosed cancer patients, and 50,000 deaths. The global cancer drug market reached $290 billion in 2020, with the cancer vaccine market valued at $5.68 billion and cell therapy at $7.8 billion. The estimated annual compound growth rate for the period 2021-2028 is projected to reach 14.5%. However, it is difficult to diagnosis tumor in an early stage.

Tumor neoantigens are fragments generated during the growth of tumor cells, exhibiting tumor specificity. As so far, none of current neoantigens identification methods are effective and well differentiate different kinds of neoantigens. As a result, there is a continuing need to develop an efficient solution or process for tumor diagnosis.

### SUMMARY OF THE INVENTION

In view of the abo-v background of the invention and to meet the requirements of the industry, the invention discloses a method for establishing a tumor neoantigen database and processes for identifying the mutated tumor-specific antigens (mTSAs) and aberrantly expressed tumor-specific antigens (aeTSAs), respectively.

Herein, the disclosed method and process for establishing a tumor neoantigen database and processes for identifying the mutated tumor-specific antigens (mTSAs) and aberrantly expressed tumor-specific antigens (aeTSAs) are executed in computers. Tools or software are applied according to requirements or purposes of each step described in the method and process, but not limited to their version or name.

Generally, genetic instability of cancer cells often generates abundant somatic mutations, and these non-synonymous variations can produce mutated tumor-specific antigens (mTSAs) which referred to as neoantigens. In addition to mutation-derived neoantigens, aberrantly expressed TSAs (aeTSAs), arising from epigenetic changes and cis- or trans-acting genetics, are also prospective materials for cancer immunotherapy and referred to as neoantigens. Since these neoantigens are highly immunogenic, they can activate T cells to trigger an immune response. In order to identify and differentiate mTSAs and aeTSAs, the invention provides a comprehensive method to establish a tumor neoantigen database and processes for identifying both mTSAs and aeTSAs in a subject, respectively.

In one aspect, the method for establishing a tumor neoantigens database comprises steps of executing a process for building a mutated tumor-specific antigens (mTSAs) dataset and a process for building an aberrantly expressed tumor-specific antigens (aeTSAs) dataset, respectively; and integrating the mutated tumor-specific antigens (mTSAs) dataset and the aberrantly expressed tumor-specific antigens (aeTSAs) dataset in a database for establishing the tumor neoantigen database.

Typically, processes for building a tumor neoantigens database, which started by extracting and sequencing DNA or RNA from tumor and adjacent normal tissues in vitro. Different workflows are followed based on the type of input data. While DNA-seq was provided, the process began with the identification of mTSAs by translating missense mutations and flanking nucleotide bases into peptides. Once RNA-seq was provided as well, gene expression quantification would be performed to filter out peptides with low expression. Similarly, mTSAs are also identified with RNA-seq data. To recognize aeTSAs, an alignment-free approach was employed, which involved retaining short k-mer sequences observed only in tumor tissues, assembling and translating them into longer peptide sequences. LC-MS/MS peptides are included to enhance the confidence of the results. Predictions of binding affinities between peptides and major histocompatibility complexes (MHC) molecules were made. When a tumor-specific peptide was anticipated to be present on the MHC molecules on cell surfaces, it was designated as a putative neoantigen. The tumor neoantigens database also included the IEDB, COSMIC (Catalogue of Somatic Mutations in Cancer), and other similar databases, associated with cancer proteomics and common mutated tumor genes, respectively.

To identify potential mTSAs, somatic mutations are discovered and annotated. Afterward, missense mutations and flanking nucleotide bases are further translated into peptides. Quantification of gene expression is conducted to filter out those peptides with low expression levels if RNA-seq reads are provided as well.

To identify aeTSAs, most of which originate from allegedly noncoding regions, an alignment-free approach is employed. The RNA-seq reads from tumor and normal tissues are chopped into short k-mer sequences. Those presenting in tumors but not normal tissues are kept, assembled into longer sequences, and translated into peptides then. LC-MS/MS peptides are optionally provided to improve the confidence of the results. Binding affinities between specific major histocompatibility complexes (MHC) and all translated peptides are predicted. Generally, if a tumor-specific peptide can successfully bind with the MHC on the surfaces of tumor cells, it will be defined as a potential neoantigen candidate.

In another aspect, to predict the binding affinity between neoantigen candidates and MHC molecules in a subject, it was essential to determine the HLA genotypes prior to performing neoantigen identification. This approach facilitated the development of personalized cancer immunotherapies or vaccines customized to each HLA genotypes. This study utilized paired-end DNA or RNA reads from normal samples for prediction. The first step involved quality control (QC) of paired-end DNA-seq or RNA-seq data using FastQC (version 0.11.9) and MultiQC (version 1.13) or similar software for the quality control. The poor quality sequences and adaptors were then trimmed using Trimmomatic (version 0.39) or similar software for reads trimming. Finally, HLA genotyping of samples was performed using a high-resolution HLA genotyping tool that predicts a sample's HLA alleles using DNA-seq or RNA-seq data.

In another aspect, the process for identifying mutated tumor-specific antigens (mTSAs) in a subject is provided in the invention.

Generally, the process of mTSAs identification with DNA-seq encompassed the following procedures. Firstly, paired-end DNA-seq of both tumor and adjacent normal samples were executed using the Illumina platforms. Trimmomatic (version 0.39) or similar software was employed to trim sequences with poor quality and remove adaptors from raw reads, while quality control of raw and trimmed reads were assessed via FastQC (version 0.11.9) and MultiQC (version 1.13) or similar software. Then, the cleaned reads were aligned to the GRCh38 (hg38) human reference genome (Ensembl Release v108) using BWA (version 0.7.17) or similar software for somatic mutations and DRAGMAP (version 1.2.1) or similar software for germline mutations. Finally, the output BAM files were sorted, and duplicates were marked with Picard (version 2.27.4) or similar software for refining the mutation calling procedure. After that, we offered two distinct pipelines to detect germline mutations and somatic mutations, respectively, in the variant calling process.

To detect germline mutations, the sorted BAM files were pre-processed by applying the tool named “gatk CalibrateDragstrModel” function from GATK4 (version gatk4.2) or similar tool, which estimated the parameters for the DRAGstr model. Subsequently, the tool named “gatk HaplotypeCaller” or similar tool performed the variant calling via local reassembly of haplotypes for germline single nucleotide polymorphisms (SNPs) and insertions and deletions (INDELs). Finally, the tools named “gatk VariantFiltration” and “gatk SelectVariants” were applied to filter out variants with lower quality scores.

To call somatic single nucleotide variants (SNVs) and INDELs, the tool named GATK4 Mutect2 pipeline or similar pipelines were employed, which performed local haplotype assembly. To remove any potential contaminations, the tool named “gatk LearnReadOrientationModel” or similar tools were applied to estimate the maximum likelihood of artifact prior probabilities in the orientation bias mixture model filter. In addition, the tool named “gatk GetPileupSummaries” or similar tools were used to tabulate pileup metrics for inferring contamination. Lastly, somatic SNVs and were filtered using the tools named “gatk FilterMutectCalls” and “gatk SelectVariants” or other tools with the similar functions.

Once germline and somatic mutations were identified, the tool named “gatk ReadBackedPhasing” function in GATK3 (version 3.8) or similar software was employed to phase SNVs using read overlap information to call variants. Herein, physical phasing was used as a robust genomic methodology to discern and allocate genetic variants to specific haplotypes within an individual's genome. This enabled generating a phased Variant Call Format (VCF) file with correct haplotypes of proximal germline mutations that could alter peptide sequences of mTSAs and influence the accuracy of binding affinities.

The subsequent step involved utilizing the Ensembl Variant Effect Predictor (VEP) or similar software for variant annotation. This process was crucial in enabling the prioritization of variants that were likely to have functional significance or relevance to a specific phenotype. VEP is a Linux-based tool that facilitates the prediction of the functional effects of genetic variants and the annotation of different types of genetic variants such as SNPs, INDELs, and copy number variants. VEP provides valuable information regarding the variant's position in the genome, the genes it impacts, and the potential consequences of the variant on protein structures and functions. The output from VEP included both VCF and Plain Text (TXT) files consisting of annotated variants.

While LC-MS/MS peptides were provided for result verification, a peptide database was generated using BLAST+ or similar software for comparing peptides that were present in both mTSA pools and LC-MS/MS results. Subsequently, pVAC-Seq or similar tool was utilized to predict the binding affinity between MHC and identified mTSA candidates. The predicted IC₅₀ value (nm) was automatically filtered by the pVAC-Seq to obtain acceptable peptide candidates, which could be further refined by adjusting the binding filter threshold based on user requirements. By default, the IC₅₀ threshold for acceptable mTSA candidates was set to 500 nm. The binding affinity filter categorized peptides as “strong binding” if their IC₅₀ values were lower than 50 nm, “intermediate binding” if between 50 nm and 250 nm, and “weak binding” if between 250 nm and 500 nm. Upon completion of the analysis, TSV and FASTA files with identified mTSAs from DNA-seq would be generated and provided to the users as final reports.

The process of mTSA identification with RNA-seq, which includes the following procedures. At first, RNA-seq reads from tumor and adjacent normal samples were sequenced by Illumina platforms. Trimmomatic (version 0.39) or similar tool was utilized to trim bad quality reads and remove adapters, and FastQC (version 0.11.9) and MultiQC (version 1.13) or similar tool could provide quality control of raw and trimmed reads. Next, the mapping of the reads to the GRCh38 (hg38) human reference human genome was performed by STAR (version 2.7.10a) or similar tool. The output BAM files were sorted, and duplicates were marked with Picard (version 2.27.4) or similar software for refining the somatic mutation calling procedure.

To minimize the potential for contamination, a tool named “gatk AddOrReplaceReadGroups” or other similar software was employed to assign all reads in a given file to a new read-group. Additionally, another tool named “gatk SplitNCigarReads” or similar software was used to split any reads that contain Ns in their concise idiosyncratic gapped alignment report (CIGAR) strings. Finally, tools named “gatk BaseRecalibrator” and “gatk ApplyBQSR” or similar software could generate base quality score recalibration (BQSR) table and apply BQSR. Afterward, somatic mutations were identified using VarScan2 or similar tools. The pileup subcommand of the identical tool was applied to the output of BQSR BAM file in order to compare the aligned reads with the reference genome. VarScan2 or similar tools then analyzed the output of the pileup format and determined whether each base met the given threshold for a significant variant. Similar to the pipeline of mTSA identification from DNA-seq, a tool named “gatk LearnReadOrientationModel” was implemented to estimate the maximum likelihood of artifact prior probabilities in the orientation bias mixture model filter. Additionally, another tool named “gatk GetPileupSummaries” was used to generate pileup metrics to infer contamination. Somatic mutations identified by VarScan2 were subjected to filtering via tools named “gatk FilterMutectCalls” and “gatk SelectVariants”. These tools mentioned above can be replaced by others with a similar function. Finally, VEP or similar software was utilized for comprehensive annotation of somatic variants. This critical process enabled the prioritization of variants that were likely to have functional significance or relevance to a specific phenotype. The annotated somatic mutations were translated into amino acid sequences using in-house scripts.

Afterward, transcript expression levels were quantified with Kallisto or similar software and the results were expressed as transcripts per million (TPM). Neoantigens candidates having a tumor expression level of less than 1 TPM were excluded from consideration. To confirm the results obtained from LC-MS/MS peptides, COSMIC, or other databases, users might provide their own peptides for verification. To enable comparison between the LC-MS/MS peptides and those presenting in mTSA pools, a peptide database was generated using BLAST+ or similar software. In a similar manner to the pipeline used for mTSA identification from DNA-seq data, pVAC-Seq or similar software was employed to predict the binding affinity between MHC and the mTSA candidates identified from RNA-seq data. The binding affinity filter was applied to categorize peptides as “strong binding” if their IC₅₀ values are below 50 nm, “intermediate binding” if between 50 nm and 250 nm, and “weak binding” if between 250 nm and 500 nm. Once the analysis was completed, final TSV and FASTA files with the identified mTSAs from RNA-seq were generated and provided to the users.

In still another aspect, a comprehensive approach for identifying aeTSAs using RNA-seq data in a subject is disclosed. This approach comprised several steps, including quality control and adaptor trimming, which were identical to those utilized for mTSA identification. Next, an alignment-free approach called k-mer profiling was utilized to detect peptides from both coding and noncoding regions. This approach allowed for the identification of peptides encoded by any reading frame from any genomic source, including structural variants. Initially, short k-mer sequences were generated from RNA-seq reads from both tumor and normal tissues using Jellyfish or similar software. The short k-mer unique sequences in tumor tissues were retained and assembled into longer sequences. Following the translation of the resulting sequences into peptides in three frames through an in-house script, the identified aeTSA candidates were subjected to the same pipeline utilized for mTSA identification. Ultimately, the final reports of mTSAs and aeTSAs were generated, which included a recommendation system based on MHC binding affinities and stability scores for each peptide

Once the tBLASTn or similar software search was conducted, the pipeline proceeded to generate a summary table for aeTSAs. The table provided valuable information such as read counts and biotype classification, cDNA sequences, and inferred coordinates of the genome from which the aeTSAs originate. The summary table included a classification of peptides into different types based on specific criteria. Peptides originating from the immunoglobulin gene (IG gene) were excluded from consideration as aeTSAs or tumor-associated antigens (TAAs), which showed substantially higher expression levels in cancer cells than in normal cells. After removing peptides derived from the IG gene, the expressing read counts were used to differentiate between aeTSAs and TAAs. For peptide categorization, when the expected total read counts from the tumor and normal samples were equal to or greater than ten, peptides were categorized as aeTSAs if the average ratio of read depth between tumor and normal samples was greater than ten. On the other hand, if the average ratio of read depth was between two and ten, and the peptides originated from exons, they would be classified as TAAs. This approach allowed for the identification and distinction of aeTSAs and TAAs based on their relative expression levels, providing valuable insights into their potential significance and role in tumor immunology. The annotation step could offer a comprehensive overview of the identified aeTSAs and their corresponding attributes, facilitating further analysis and interpretation of the results.

In conclusion, the invention discloses a novel method for establishing a tumor-neoantigen database. The tumor-neoantigen database comprises at least one tumor-specific neoantigens (mTSAs) dataset, at least one aberrantly expressed tumor-specific neoantigen (aeTSAs) dataset and at least one liquid chromatography-MS/MS (LC-MS/MS) dataset. The tumor-neoantigen database is used for predicting tumor variants of clinical samples, such as cell samples or tissue samples. Moreover, the processes for identifying tumor-specific neoantigens (mTSAs) and aberrantly expressed tumor-specific neoantigen (aeTSAs) in a subject are capable of using for determining tumor variants of clinical samples.

### BRIEF DESCRIPTION OF THE DRAWINGS

FIG. 1 is a process flow diagram regarding the process for building a mutated tumor-specific antigens (mTSAs) dataset from DNA sequencing reads; and

FIG. 2 is a process flow diagram regarding the process for building a mutated tumor-specific antigens (mTSAs) dataset and an aberrantly expressed tumor-specific antigens (aeTSAs) dataset from RNA sequencing reads.

### EMBODIMENTS

In a first embodiment, the invention discloses a method for establishing a tumor neoantigen database. Specifically, the tumor neoantigen database comprises at least one tumor-specific neoantigens (mTSAs) dataset, at least one aberrantly expressed tumor-specific neoantigen dataset and at least one liquid chromatography-MS/MS (LC-MS/MS) dataset.

In one embodiment, the method for establishing a tumor neoantigen database comprises steps of executing a process for building a mutated tumor-specific antigens (mTSAs) dataset and a process for building an aberrantly expressed tumor-specific antigens (aeTSAs) dataset, respectively; and integrating the mutated tumor-specific antigen (mTSAs) dataset and the aberrantly expressed tumor-specific antigens dataset in a database for establishing the tumor neoantigen database.

In one embodiment, the method for establishing a tumor neoantigen database further comprises a first step of extracting DNA or RNA from clinical samples in vitro. The clinical samples comprise cell samples or tissue samples.

In one embodiment, the process for building a mutated tumor-specific antigens (mTSAs) dataset comprises following steps.

Execute paired-end DNA or RNA sequencing of clinical samples to obtain raw paired end DNA or RNA sequencing reads; trim and remove adaptors of the raw paired-end DNA or RNA sequencing reads to obtain trimmed paired-end DNA or RNA sequencing reads; map the trimmed paired-end DNA or RNA sequencing reads to reference genome to output BAM files include somatic mutations and germline mutations; mark duplicates to identify and label duplicated items that may occur during analysis for ensuring that redundant data is not counted multiple times in subsequent steps; perform variant calling to identify the somatic mutations and germline mutations; perform variants phasing to locate their original genes and understand how these variations are inherited and their relationships with each other; estimate transcript expression levels and filtering out expressed variants with a tumor expression level of lower than 1 transcripts per million (TPM); translate the expressed variants with a tumor expression level of more than 1 TPM to corresponding peptides; mark the corresponding peptides found in LC-MS/MS databases, which represent peptides sequenced from cell surface proteins, to enhance credibility in identifying mTSA candidates; and predict binding affinity levels between major histocompatibility complex (MHC) and the mTSA candidates, and when IC₅₀ values are less than or equal to 500, the mTSA candidates are identified as mTSAs and stored in a dataset.

In one example of the embodiment, the reference genome is hg38 human reference genome.

In one example of the embodiment, the tumor neoantigen database is used for predicting tumor variants of clinical samples.

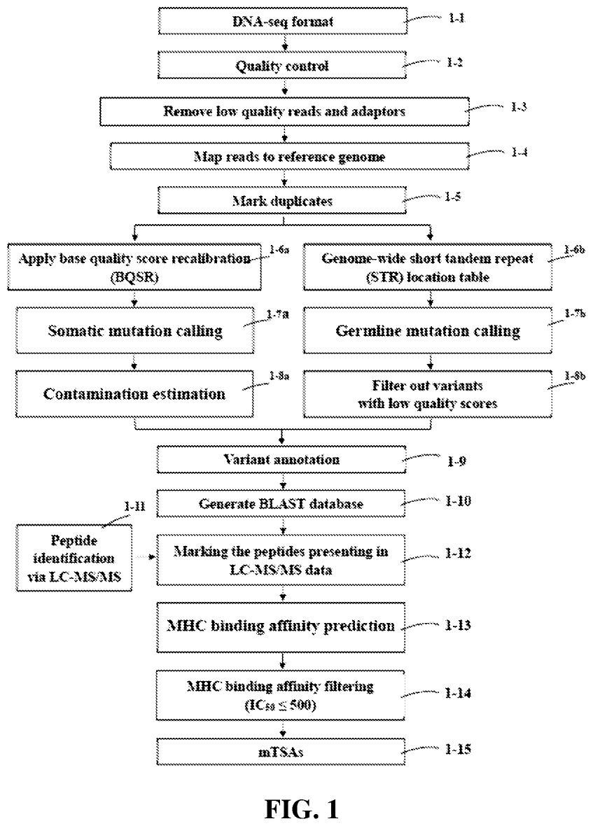

In one representative example of the embodiment of building a mutated tumor-specific antigens(mTSAs) dataset from DNA sequencing reads, please refer to FIG. 1. Step 1-1 is to provide a DNA sequence format (FASTAQ); step 1-2 is to perform quality control of the DNA sequence format by tools such as FastQC and MultiQC; step 1-3 is to remove low quality reads and adaptors by a tool such as Trimmomatic to obtain trimmed reads; step 1-4 is to map the trimmed reads to reference genome by tools such as BWA/DRAGMAP; step 1-5 is to mark duplicates by a tool such as Picard; step 1-6a is to apply base quality score recalibration (BQSR) by a tool such as BaseRecalibrator; step 1-6b is to generate genome-wide short tandem repeat (STR) location table by a tool such as DRAGEN; step 1-7a is to perform somatic mutation calling by a tool such as GATK Mutect2 after the step 1-6a; step 1-7b is to perform germline mutation calling by a tool such as GATK HaplotypeCaller; step 1-8a is to perform contamination estimation by a tool such as GATK Mutect2 after the step 1-7a; step 1-8b is to filter out variants with low quality scores by a tool such as GATK VariantFiltration; step 1-9 is to perform variant annotation by a tool such as VEP; step 1-10 is to generate BLAST database; step 1-11 is to perform peptide identification via LC-MS/MS; step 1-12 is to mark the peptides presenting in the LC-MS/MS data by a tool such as BLAST+; step 1-13 is to perform MHC binding affinity prediction by a tool such as pVACtools; step 1-14 is to perform MHC binding affinity filtering and step 1-15 is to identify mTSAs when IC₅₀ values are less than or equal to 500 and subsequently stored in a dataset.

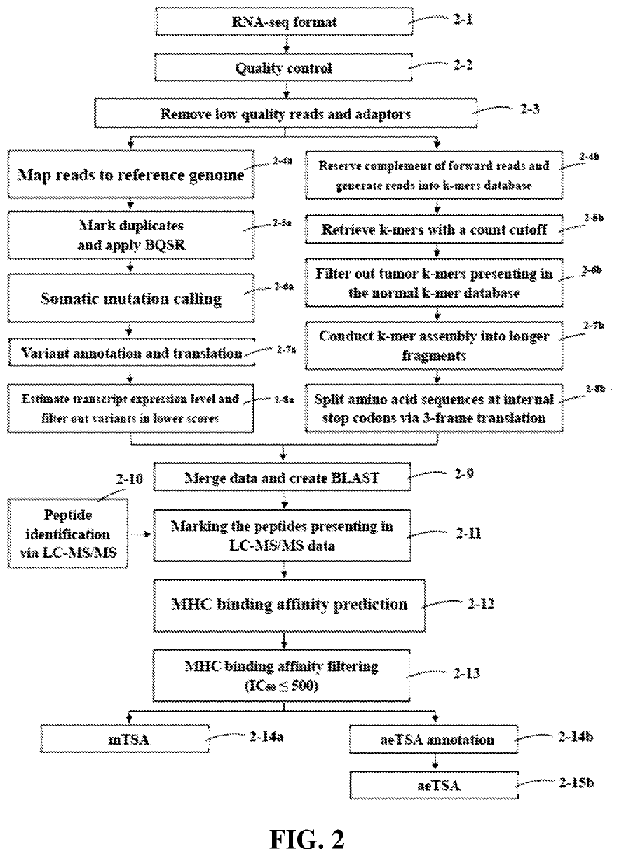

In another representative example of the embodiment of building a mutated tumor-specific antigens (mTSAs) dataset from RNA sequencing reads, please refer to FIG. 2. Step 2-1 is to provide a RNA sequence format (FASTAQ); step 2-2 is to perform quality control of the RNA sequence format by tools such as FastQC and MultiQC; step 2-3 is to remove low quality reads and adaptors by a tool such as Trimmomatic to obtain trimmed reads; step 2-4a is to map the trimmed reads to reference genome by a tool named such as STAR; step 2-5a is to mark duplicates and apply BQSR by a tool such as Picard and GATK; step 2-6a is to perform somatic mutation calling by a tool such as GATK Mutect2; step 2-7a is to perform variant annotation and translation; step 2-8a is to estimate transcript expression level and filter out variants in lower scores; step 2-9 is to merge data and create BLAST database; step 2-10 is to perform peptide identification via LC-MS/MS; step 2-11 is to mark the peptides presenting in the LC-MS/MS data by a tool such as BLAST+; step 2-12 is to perform MHC binding affinity prediction by a tool such as pVACtools; step 2-13 is to perform MHC binding affinity filtering and step 2-14a is to identify mTSAs when IC₅₀ values are less than or equal to 500 and subsequently stored in a dataset.

In another embodiment, the process for building an aberrantly expressed tumor-specific antigens (aeTSAs) dataset comprises following steps.

Execute paired-end RNA sequencing of clinical samples to obtain raw paired-end RNA sequencing reads; trim and remove adaptors of the raw paired-end RNA sequencing reads to obtain trimmed paired-end RNA sequencing reads; reverse forward reads to obtain their complement, and these sequences are then fragmented into smaller units known as k-mers; filter out the k-mers with a count below a certain threshold to ensure that only those meeting the specified criteria for quantity are included for further analysis steps; assemble the k-mers into longer fragments which represent an amino acid sequence; translate the amino acid sequence to peptide through 3-frame translation, and divided at internal stop codons; identify and select the peptide found in LC-MS/MS databases, which represent the peptides sequenced from cell surface proteins, to enhance credibility in identifying aeTSA candidates; predict binding affinity levels between major histocompatibility complex (MHC) and the aeTSA candidates, and retain the aeTSA candidates when their IC₅₀ values are less than or equal to 500; and perform aeTSA annotation to determine the gene origins of each aeTSA candidate, with the definition that those exhibiting sufficient transcript read counts will be classified as aeTSA and subsequently stored in a dataset.

In one representative example of another embodiment of building an aberrantly expressed tumor-specific antigens (aeTSAs) dataset from RNA sequencing reads, please refer to FIG. 2. Step 2-1 is to provide a RNA sequence format (FASTAQ); step 2-2 is to perform quality control of the RNA sequence format by tools such as FastQC and MultiQC; step 2-3 is to remove low quality reads and adaptors by a tool such as Trimmomatic to obtain trimmed reads; step 2-4b is to reserve complement of forward reads and generate reads into k-mers database; step 2-5b is to retrieve the k-mers with a count cutoff; step 2-6b is to filter out tumor k-mers presenting in the normal k-mers database; step 2-7b is to conduct k-mer assembly into longer fragments; step 2-8b is to spilt amino acid sequences at internal stop codons via 3-frame translation; step 2-9 is to merge data and create BLAST database; step 2-10 is to perform peptide identification via LC-MS/MS; step 2-11 is to mark the peptides presenting in the LC-MS/MS data by a tool such as BLAST+; step 2-12 is to perform MHC binding affinity prediction by a tool such as pVACtools; step 2-13 is to perform MHC binding affinity filtering; step 2-14b is to perform aeTSAs annotation and step 2-15b is to identify asTSA with the definition that those exhibiting sufficient transcript read counts and subsequently stored in a dataset.

In one example, we applied the online system having the tumor neoantigen database to identify 95 putative aeTSA candidates sharing among 13 patients with colorectal cancer, and on top of that 14 of them could be presented by HLA-A*11:01 & 11:02, which are common alleles in Asians. We also compared 246 putative mTSA candidates with peptides in COSMIC database and found 15 of them were common cancer variants, including KRAS and BRAF mutations, both of which are prognostic and predictive biomarkers in colorectal cancer. More importantly, initial evidence shows that these candidates are immunogenic on primary peripheral blood mononuclear cells. Accordingly, the online system having the tumor neoantigen database is a very useful and suitable to identify and differentiate mTSAs and aeTSAs. It integrates analysis results of various inputs, i.e., DNA sequencing, RNA sequencing, and LC-MS/MS data, which can improve the reliability of identified TSAs and provide valuable information for clinical investigators.

In another embodiment, a process for identifying a mutated tumor-specific antigens (mTSAs) in a subject is disclosed. The process for identifying a mutated tumor-specific antigens (mTSAs) in a subject comprises following steps.

Extract DNA or RNA from a subject in vitro; execute paired-end DNA or RNA sequencing of the subject to obtain raw paired end DNA or RNA sequencing reads; trim and remove adaptors of the raw paired-end DNA or RNA sequencing reads to obtain trimmed paired-end DNA or RNA sequencing reads; map the trimmed paired-end DNA or RNA sequencing reads to hg38 human reference genome to output BAM files include somatic mutations and germline mutations; mark duplicates to identify and label duplicated items that may occur during analysis for ensuring that redundant data is not counted multiple times in subsequent steps; perform variant calling to identify the somatic mutations and germline mutations; perform variants phasing to locate their original genes and understand how these variations are inherited and their relationships with each other; estimate transcript expression levels and filtering out expressed variants with a tumor expression level of lower than 1 transcripts per million (TPM); translate the expressed variants with a tumor expression level of more than 1 TPM to corresponding peptides; mark the corresponding peptides found in LC-MS/MS databases, which represent peptides sequenced from cell surface proteins, to enhance credibility in identifying mTSA candidates; and predict binding affinity levels between major histocompatibility complex (MHC) and the mTSA candidates, and when IC₅₀ values are less than or equal to 500, the mTSA candidates are identified as mTSAs in the subject.

In one example, the subject comprises cell samples or tissue samples.

In still another embodiment, a process for identifying an aberrantly expressed tumor-specific antigens (aeTSAs) in a subject is disclosed. The process for identifying an aberrantly expressed tumor-specific antigens (aeTSAs) in a subject comprises following steps.

Extract RNA from a subject in vitro; execute paired-end RNA sequencing of clinical samples to obtain raw paired-end RNA sequencing reads; trim and remove adaptors of the raw paired-end RNA sequencing reads to obtain trimmed paired-end RNA sequencing reads; reverse forward reads to obtain their complement, and these sequences are then fragmented into smaller units known as k-mers; filter out the k-mers with a count below a certain threshold to ensure that only those meeting the specified criteria for quantity are included for further analysis steps; assemble the k-mers into longer fragments which represent amino acid sequences; translate the amino acid sequences to peptides through 3-frame translation, and divided at internal stop codons; identify and select the peptides found in LC-MS/MS databases, which represent peptides sequenced from cell surface proteins, to enhance credibility in identifying aeTSA candidates; predict binding affinity levels between major histocompatibility complex (MHC) and the aeTSA candidates, and retain the aeTSA candidates when their IC₅₀ values are less than or equal to 500; and perform aeTSA annotation to determine the gene origins of each aeTSA candidate, with the definition that those exhibiting sufficient transcript read counts will be classified as aeTSA in the subject.

In one example, the subject comprises cell samples or tissue samples.

In conclusion, the invention provides a comprehensive method for establishing a tumor neoantigens database and a process for identifying mTSAs and aeTSAs from DNA sequencing, RNA sequencing and liquid chromatography-MS/MS (LC-MS/MS) data, respectively.

Obviously, many modifications and variations are possible in the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method for establishing a tumor neoantigen database, comprising,
executing a process for building a mutated tumor-specific antigens (mTSAs) dataset and a process for building an aberrantly expressed tumor-specific antigens (aeTSAs) dataset, respectively; and
integrating the mutated tumor-specific antigen (mTSAs) dataset and the aberrantly expressed tumor-specific antigens dataset in a database for establishing the tumor neoantigen database.

2. The method for establishing a tumor neoantigen database of claim 1, further comprises a first step of extracting DNA or RNA from clinical samples.

3. The method for establishing a tumor neoantigen database of claim 1, wherein the process for building a mutated tumor-specific antigens (mTSAs) dataset comprises following steps:
executing paired-end DNA or RNA sequencing of clinical samples to obtain raw paired end DNA or RNA sequencing reads;
trimming and removing adaptors of the raw paired-end DNA or RNA sequencing reads to obtain trimmed paired-end DNA or RNA sequencing reads;
mapping the trimmed paired-end DNA or RNA sequencing reads to reference genome to output BAM files include somatic mutations and germline mutations;
marking duplicates to identify and label duplicated items that may occur during analysis for ensuring that redundant data is not counted multiple times in subsequent steps;
performing variant calling to identify the somatic mutations and germline mutations;
performing variants phasing to locate their original genes and understand how these variations are inherited and their relationships with each other;
estimating transcript expression levels and filtering out expressed variants with a tumor expression level of lower than 1 transcripts per million (TPM);
translating the expressed variants with a tumor expression level of more than 1 TPM to corresponding peptides;
marking the corresponding peptides found in LC-MS/MS databases, which represent peptides sequenced from cell surface proteins, to enhance credibility in identifying mTSA candidates; and
predicting binding affinity levels between major histocompatibility complex (MHC) and the mTSA candidates, and when IC₅₀ values are less than or equal to 500, the mTSA candidates are identified as mTSAs and stored in a dataset.

4. The method for establishing a tumor neoantigen database of claim 3, wherein the reference genome is hg38 human reference genome.

5. The method for establishing a tumor neoantigen database of claim 1, wherein the process for building an aberrantly expressed tumor-specific antigens (aeTSAs) dataset comprises following steps:
executing paired-end RNA sequencing of clinical samples to obtain raw paired-end RNA sequencing reads;
trimming and removing adaptors of the raw paired-end RNA sequencing reads to obtain trimmed paired-end RNA sequencing reads;
reversing forward reads to obtain their complement, and these sequences are then fragmented into smaller units known as k-mers;
filtering out the k-mers with a count below a certain threshold to ensure that only those meeting the specified criteria for quantity are included for further analysis steps;
assembling the k-mers into longer fragments which represent an amino acid sequence;
translating the amino acid sequence to peptide through 3-frame translation, and divided at internal stop codons.
identifying and selecting the peptide found in LC-MS/MS databases, which represent the peptide sequenced from cell surface proteins, to enhance credibility in identifying aeTSA candidates.
predicting binding affinity levels between major histocompatibility complex (MHC) and the aeTSA candidates, and retaining the aeTSA candidates when their IC₅₀ values are less than or equal to 500; and
performing aeTSA annotation to determine the gene origins of each aeTSA candidate, with the definition that those exhibiting sufficient transcript read counts will be classified as aeTSA and subsequently stored in a dataset.

6. The method for establishing a tumor neoantigen database of claim 1, wherein the tumor neoantigen database is used for predicting tumor variants of clinical samples.

7. A process for identifying a mutated tumor-specific antigens (mTSAs) in a subject, comprising,
extracting DNA or RNA from a subject;
executing paired-end DNA or RNA sequencing of the subject to obtain raw paired end DNA or RNA sequencing reads;
trimming and removing adaptors of the raw paired-end DNA or RNA sequencing reads to obtain trimmed paired-end DNA or RNA sequencing reads;
mapping the trimmed paired-end DNA or RNA sequencing reads to hg38 human reference genome to output BAM files include somatic mutations and germline mutations;
marking duplicates to identify and label duplicated items that may occur during analysis for ensuring that redundant data is not counted multiple times in subsequent steps;
performing variant calling to identify the somatic mutations and germline mutations;
performing variants phasing to locate their original genes and understand how these variations are inherited and their relationships with each other;
estimating transcript expression levels and filtering out expressed variants with a tumor expression level of lower than 1 transcripts per million (TPM);
translating the expressed variants with a tumor expression level of more than 1 TPM to corresponding peptides;
marking the corresponding peptides found in LC-MS/MS databases, which represent peptides sequenced from cell surface proteins, to enhance credibility in identifying mTSA candidates; and
predicting binding affinity levels between major histocompatibility complex (MHC) and the mTSA candidates, and when IC₅₀ values are less than or equal to 500, the mTSA candidates are identified as mTSAs in the subject.

8. The process for identifying a mutated tumor-specific antigens (mTSAs) in a subject of claim 7, wherein the subject comprises cell samples or tissue samples.

9. A process for identifying an aberrantly expressed tumor-specific antigens (aeTSAs) in a subject, comprising,
extracting RNA from a subject;
executing paired-end RNA sequencing of clinical samples to obtain raw paired-end RNA sequencing reads;
trimming and removing adaptors of the raw paired-end RNA sequencing reads to obtain trimmed paired-end RNA sequencing reads;
reversing forward reads to obtain their complement, and these sequences are then fragmented into smaller units known as k-mers;
filtering out the k-mers with a count below a certain threshold to ensure that only those meeting the specified criteria for quantity are included for further analysis steps;
assembling the k-mers into longer fragments which represent an amino acid sequence;
translating the amino acid sequence to peptide through 3-frame translation, and divided at internal stop codons;
identifying and selecting the peptide found in LC-MS/MS databases, which represent peptides sequenced from cell surface proteins, to enhance credibility in identifying aeTSA candidates;
predicting binding affinity levels between major histocompatibility complex (MHC) and the aeTSA candidates, and retaining the aeTSA candidates when their IC₅₀ values are less than or equal to 500; and
performing aeTSA annotation to determine the gene origins of each aeTSA candidate, with the definition that those exhibiting sufficient transcript read counts will be classified as aeTSA in the subject.

10. The process for identifying an aberrantly expressed tumor-specific antigens (aeTSAs) in a subject of claim 9, wherein the subject comprises cell samples or tissue samples.
